(19) United States
(12) Patent Application Publication
(10) Pub. No.: US 2017/0156922 A1
(43) Pub. Date: Jun. 8, 2017

(54) STUTTER CONTROL DEVICE

(71) Applicant: Hadassah Academic College, Jerusalem (IL)

(72) Inventor: Oshrat Cohen, Psagot (IL)

(21) Appl. No.: 15/325,974

(22) PCT Filed: Jul. 16, 2015
(86) PCT No.: PCT/IL2015/050736

§ 371 Date: Jan. 12, 2017

Related U.S. Application Data
(60) Provisional application No. 62/025,018, filed on Jul. 16, 2014.

(51) Int. Cl. A61F 5/58; A61H 9/00; A61H 7/00; A61H 15/00

(52) U.S. Cl. CPC A61F 5/58; A61H 15/00; A61H 9/005; A61H 7/002

(57) ABSTRACT

A device for stimulating control of stutter. The device includes a moving-sensation part, a portion of which is arranged to contact a user of the device, which is configured to exert a tactile sensation of moving over the user body in a continuous pattern, for helping users to control their flow of speech.

### FIELD OF THE INVENTION

The present invention relates to stuttering in general, and to a device for controlling speech flow in particular.

### BACKGROUND OF THE INVENTION

Stuttering is a speech disorder where the flow of speech is disrupted by involuntary repetitions and prolongations of sounds, syllables, words or phrases, as well as involuntary silent pauses in which the stutterer is unable to produce sounds. The impact of stuttering on the way a person functions and their emotional state can be severe. Stutterers may experience a fear of having to enunciate specific vowels or consonants, a fear of being caught stuttering in social situations, self-imposed isolation, anxiety, stress, or shame. Stuttering is also variable. In certain situations, such as singing, talking on the telephone or when in a large group, the stuttering might be more severe or less.

There are known speech therapy techniques that may help increase a stutterer's fluency, the ability to speak easily and smoothly. For severe stuttering, long-term therapy and hard work can be required to increase fluency. In one treatment, a patient uses a metronome, with an audio or optical signal (e.g., a flashing light) to help control and regulate their rhythm of speech. In another treatment the patient is instructed to manually tap on his leg or elsewhere.

### SUMMARY OF THE INVENTION

In accordance with one aspect of the present invention, there is thus provided a device for stimulating a user in order to control speech stutter. The device includes a moving sensation part, a portion of which is arranged to contact a user of the device, which is configured to exert a tactile sensation of a moving over the user's body in a continuous pattern, for helping users to control their flow of speech. The path may be a reciprocating (side-to-side, back-and-forth, linear) pattern, a closed curved path, an open curved path, a wave path, a figure-eight path, an ellipsoidal path, a helical path, a spiraling path, a closed polygonal path, an open polygonal path, a changing path, or a random path. The speed of the continuous pattern may be in a steady speed pattern, a varying speed pattern, varying according to a predetermined program, or a randomly varying speed pattern. The pressure of the continuous pattern may be in a steady pressure pattern, a varying pressure pattern, varying according to a predetermined program, or a randomly varying pressure pattern. The path of the moving sensation part may be in a fixed location, wherein the moving sensation is created by gradual pressure change, gradual speed change; or an alternating rolling direction of a rolling element of the moving sensation part.

The moving sensation part may include a rolling element in rolling contact with the user which is moved along the path. The rolling element can rotate about an asymmetrical axis to create a dynamically changing pressure over a portion of the user body. The rolling element may be lubricated over its tactile interfacing surface for reducing skin drag and irritation. The rolling element may include a cross sectional profile of a round profile, an ellipsoidal profile, an aspheric profile, a smooth closed curve profile, a closed convex curve profile, a closed curve profile whose curvature has no cusps, valleys or depressions that interrupt contact with the skin, or a unevenly curved profile.

The controller is configure to control operations including the speed of the moving sensation part, the pressure of the moving sensation part, the path of the moving sensation part, a predetermined speed program selection of the moving sensation part, a predetermined pressure program selection of the moving sensation part, a predetermined path program selection of the moving sensation part, storing usage data, recording user speech, analyzing stored or recorded data, recording user performance in conjunction with usage data, displaying stored, recorded or analyzed data, or communicating with external devices. User control interface may be used for managing at least one of the above operations.

The moving sensation part may include a gentle-stroke element in caressing contact with the user skin which is moved along the path, which may incorporate a brush. The moving sensation part may include an air jet element operative for blowing continuous air jet over the user's skin, wherein the air jet element is moved along the path. The moving sensation part may include a matrix of stationary touch sensation elements in fixed locations, wherein the touch sensation elements are selectively activated along the path to create a moving sensation. The stationary touch sensation elements may include gentle stroke, air jet, rolling, bulging, inflating, or pressure exerting elements.

The continuous pattern may be repeated at a frequency ranging from 240-12 CPM, 120-20 CPM, or 60-30 CPM, wherein each cycle may be considered by completing a closed path or back and forth movement along a section of an open path, completing a speed change cycle, or and completing a pressure change cycle.

The device may further include voice recognition for identifying when the user talks and wherein the device is controlled to be activated only when the user talks.

The device may further include at least one fastening element for attaching the device to the user. The at least one fastening element may be operative for attaching the device to the user in different body regions, and may include a clip, a belt, a removable skin-adhesive, a removable cloth-adhesive, or a band.

The moving part may be incorporated into a mobile object such as a watch or a handheld communication or interface device.

### BRIEF DESCRIPTION OF THE DRAWINGS

The present invention will be understood and appreciated more fully from the following detailed description taken in conjunction with the drawings in which:

FIG. 1 is a front view of a stutter control device, constructed and operative in accordance with an embodiment of the present invention;

FIG. 2 is a back view of the stutter control device of FIG. 1;

FIG. 3 is an illustration of an exemplary motion, caused by continuous friction, of a stutter control device, in accordance with an embodiment of the present invention;

FIGS. 4a to 4c illustrate examples of alternative tactile interfaces of the embodiment of FIG. 3;

FIGS. 5a to 5d illustrate examples of tactile interfaces for inspiring touch or movement sensation, constructed and operative in accordance with further embodiments of the present invention;

FIG. 6 is a bottom view of a stutter control device, constructed and operative in accordance with another embodiment of the present invention;

FIG. 7 is a bottom view of a stutter control device, constructed and operative in accordance with another embodiment of the present invention;

FIG. 8 illustrates examples of paths that can be used in conjunction with a stutter control device, constructed and operative in accordance with further embodiments of the present invention; and

FIG. 9 is a block diagram of a stutter control device, constructed and operative in accordance with a further embodiment of the present invention.

### DETAILED DESCRIPTION OF THE EMBODIMENTS

The present invention overcomes the disadvantages of the prior art by providing a device that helps alleviate and control stuttering phenomena. The present invention provides a tactile sensation of a continuous movement, preferably along a path over the body of a user, which helps the user to control the flow of speech. The movement along a path creates a continuous sensation of dynamic caressing. The movement is preferably conducted in a pace that conveys a rhythm, in a frequencies ranging between 0.25 to 5 Seconds per Cycle (˜0.2-4 cycles/sec), either by repeating a path in a predetermined frequency, or by cycles of dynamic speed or pressure change—along a path or at a particular fixed location (in this context, the term “path” includes a fixed location). The tactile sensation of movement along a path may be created by a moving element, or by a matrix of fixed elements that are selectively activated along the path. Tactile sensation may be generated, for instance, by a rolling element, pressurizing element, gentle stroke element (such as a brush), or an air jet streamer.

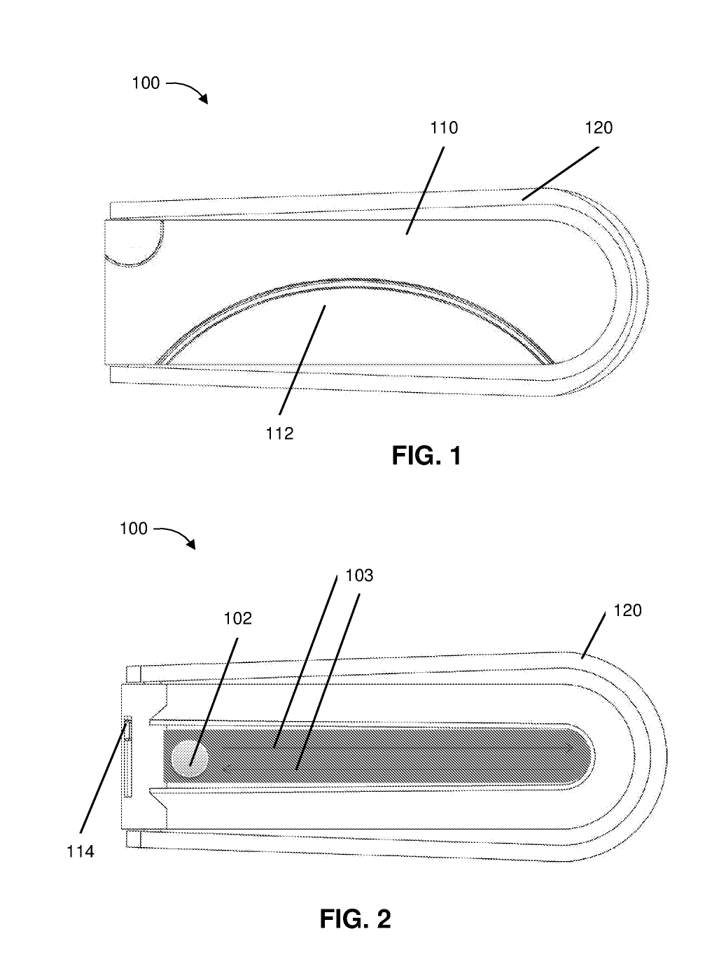

Reference is now made to FIGS. 1 and 2, which are front and back views, respectively, of a device 100, in accordance with an embodiment of the present invention. Device 100 includes a moving part 102, an intensity control button 110 (crescent cut-out shaped), a speed control button 112 (crescent-shaped), a mode switch 114, a power source (not shown), a control circuit (not shown), and a clip 120. Moving part 102, buttons 110, 112 and switch 114 are all electrically connected to the control circuit and the power source.

When device 100 is placed over the user's body, moving part 102 protrudes outwardly from device 100 towards the user so that at least a portion of moving part 102 is in contact with the user, either directly on the skin or through the clothing of the user. Moving part 102 is configured to move in a continuous pattern, such as a reciprocating side-to-side motion (repetitive up-and-down or back-and-forth linear motion) along arrows 103. Moving part 102 may be connected to a small motor and gear (not shown) that are arranged to move moving part 102 in a controlled manner. Moving part 102 can also include a rolling element so to be in rolling contact with the user (thereby creating a moving pressure location) rather than in frictional interface.

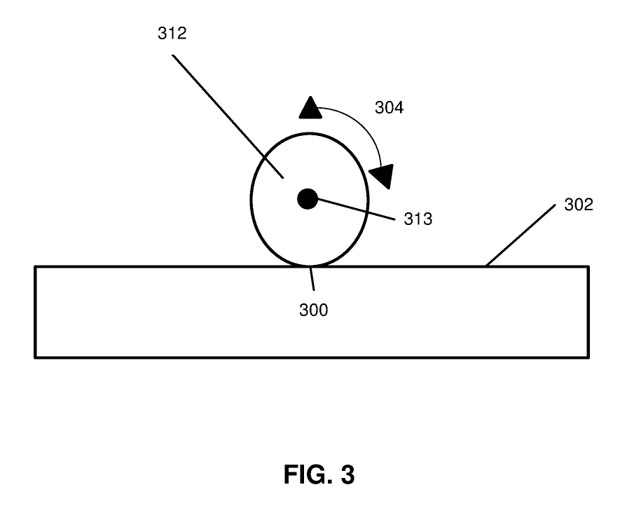

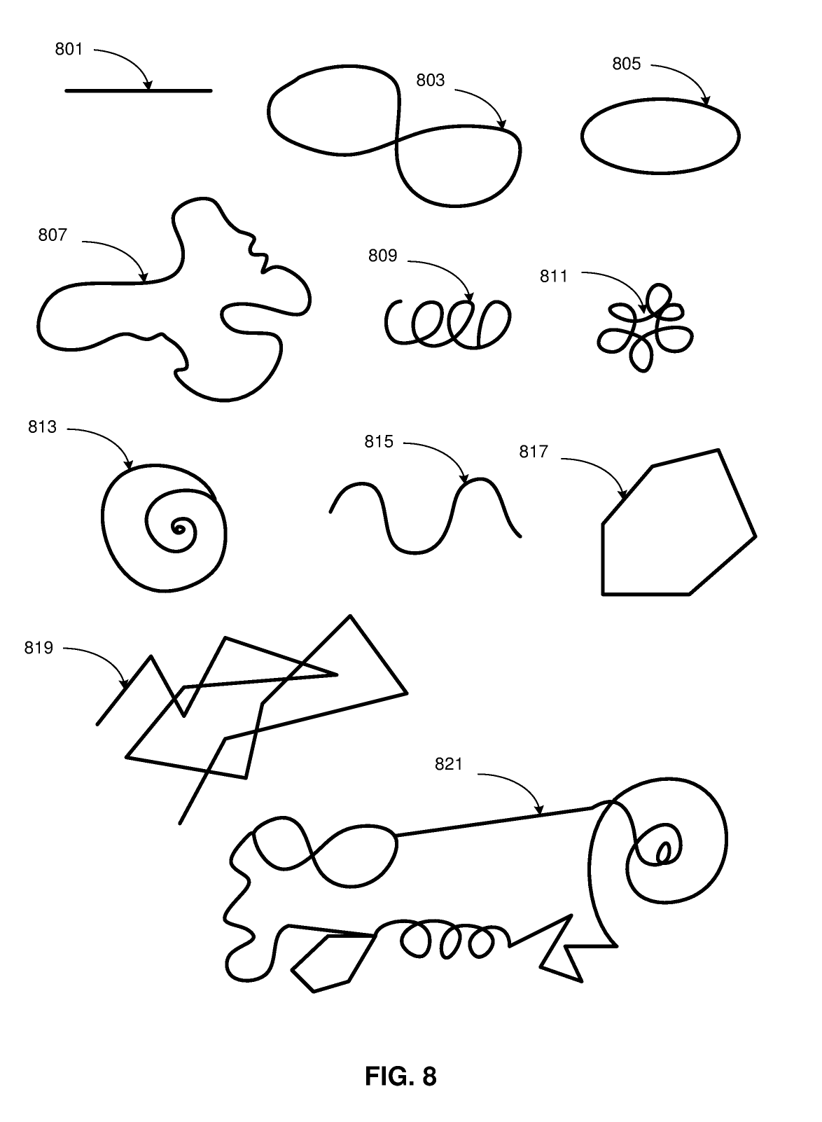

Alternatively, moving part 102 can be arranged to imitate movement using microfluidics technology. For example, a fluid channel can be routed along arrows 103 through device 100 which selectively causes a fluid to expand an expandable (and retractable) portion of a polymer layer covering the fluid channel in single location along arrows 103 to create a bulge 102. The expanded bulge location is advanced along arrows 103 so as to create movement of the expanded bulge location there along (all bulge locations along the path of arrows 103 are retracted except in a single expanded location). The typical motion of moving part 102 is reciprocating along straight path as of arrows 103. However, other motion path patterns may be provided by different paths curving over device 100, such as a wave path, a figure-eight path (lemniscate or ∞-shaped curve), a helical path, or a spiraling path (these and further examples are described below in reference to FIG. 8). Alternatively, the motion of moving part 102 can be in a fixed location such as with an eccentric sphere or ellipsoidal element as shown in FIG. 3 and described in its context below. The typical range of path length of movement of moving part 102 is between 50-70 mm, although fixed location or other lengths or distances may be applied. A typical effective motion tempo (frequency) is between 40-85 CPM (Cycles Per Minute, approximately equivalent to 0.66-1.42 cycles per second, or 1.5-0.7 seconds per cycle), although other frequencies may be used, and further examples are mentioned below.

The motion of moving part 102 is sensed by the user, whether the movement is along a path or in a single location, or whether the moving part is in frictional contact or just creates a dynamic pressure pattern. Sensing the continuous motion of moving part 102 helps the user to relax, concentrate and regulate his/her rhythm of speech. A user can appropriate or match his/her speech to the rhythm, pace, speed, or tempo of the moving part 102, e.g., saying a word or two with each stroke of the moving part. The continuous motion felt by the user is a soft flowing motion with no abrupt peaks, as opposed to a pulsing sensation. FIG. 3 is an illustration of an exemplary motion, caused by continuous friction, of a stutter control device (similar to device 100 of FIGS. 1 and 2). The stutter control device has a moving part 312 which can be a wheel (which can be a wide cylindrical rail) or ball that is disposed at a specific fixed contact point 300 on a user 302. Moving part 312 alters its direction of movement 304 at a set pace. This movement causes a continuous sensation on the user 302. When moving part 312 changes direction it is sensed by the user. The user adjusts his/her speech according to the felt sensations. If moving part 312 rotates about an off-centered axis 313 or shaped with a cross section of an eccentric sphere (such as of an ellipsoid), its rotation would also create a dynamically changing pressure over the user body. In addition moving part 312 can also be implemented to move along a path similar to moving part 102 of FIG. 1.

Referring back to FIGS. 1 and 2, a power source (not shown), such as a battery pack (or any external source), is electrically connected to the moving part 102 for providing power to the device 100. The power source can be replaceable and/or rechargeable, and device 100 can include a battery compartment and/or a power socket compatible with a power charger plug. The internal control circuit (not shown) is connected to the power source and controls the moving part 102. Intensity control button 110, speed control button 112, and mode switch 114 serve as interfaces between the user and the control circuit. Intensity control button 110 controls the force or pressure that the portion of moving part 102 in contact with the user exerts against the user such as by determining the extent by which moving part 102 protrudes from device 100. A motor and gear may be used to raise or lower moving part 102 and affect the extent to which moving part 102 protrudes outwardly from device 100 or alternatively an eccentric part may be used to allow for an adjustable pressure. A user may operate intensity control button 110 to increase or decrease the pressure according to the user's preference. Dynamically changing patterns of intensity may be preprogrammed and selected by the user. Speed control button 112 controls the speed of moving part 102. A user may operate speed control button 112 to increase or decrease the tempo according to the user's preference. Dynamically changing patterns of speed may be preprogrammed and selected by the user. Mode switch 114 allows a user to choose between a variety of modes, such as an off mode, a continual mode, and a speech activated mode. In continual mode moving part 102 moves continuously regardless of whether or not a user is speaking. In speech activated mode moving part 102 moves continuously while a user is speaking. A speaker recognition sensor (not shown) can be used to sense when a particular person, e.g., the user, is speaking and inform the control circuit that moving part 102 should be activated.

Clip 120 is arranged to help attach the device 100 to the user, for example to an article of their clothing, such as a shirt collar. In place of or in addition to clip 120, other fastening elements may be provided such as a band (e.g., headband, wristband, or leg band). Typical body placement locations of the device 100 include (but are not limited to): the arm, thigh, waist area, or others.

The operation of device 100 will now be further discussed. Device 100 is first securely and effectively attached to the user's clothes by means of clip 120 in a way that allows a portion of moving part 102 to come in contact with the user, either directly or through their clothes. The user then activates the device using mode switch 114 to select one of the active modes of the device 100 (e.g., continual mode or speech sensitive mode). The user then controls and adjusts the intensity and speed of the moving part 102 by means of intensity control button 110, and speed control button 112, according to their personal preferences.

The present invention can be used regardless of the user's language. The device of the present invention can be sized and configured in a variety of manners. It can be sized as a small, relative to the user, wearable product. Alternatively, it can be sized as a tabletop version for household and/or clinic treatment use (for practicing at home or while being guided by a therapist, for example). The present invention may be incorporated for use with a wristwatch or a handheld device, such as a mobile phone. Also, the moving element may be incorporated within a flexible surface that feels comfortable to the user when placed in contact with user body.

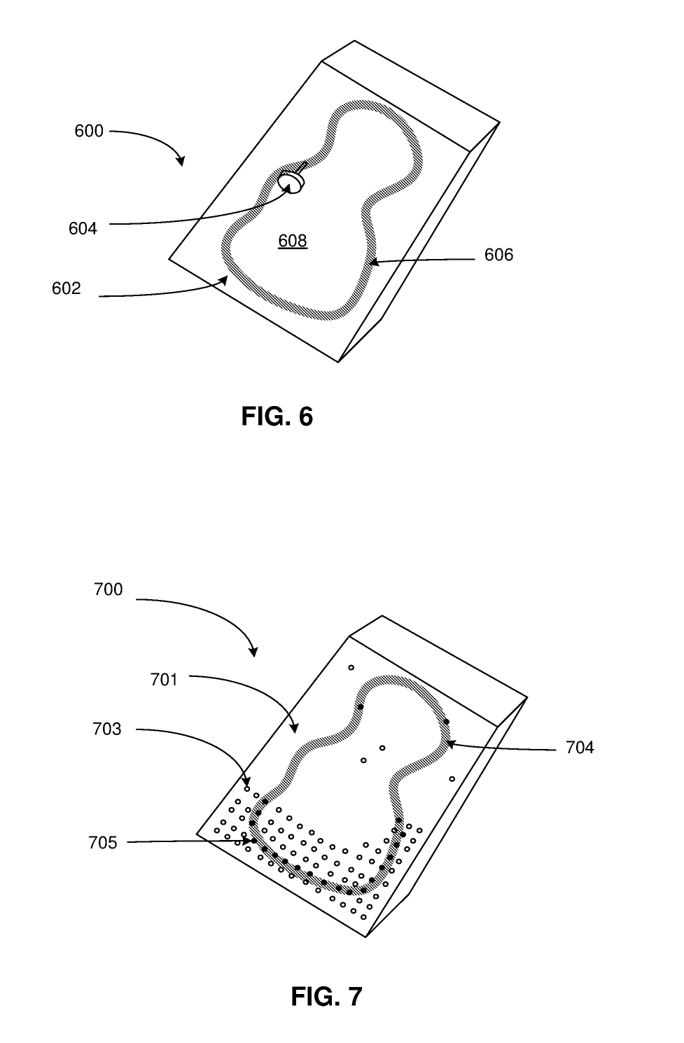

Reference is now made to FIG. 6. FIG. 6 is a bottom view of a stutter control device 600, constructed and operative in accordance with another embodiment of the present invention. Device 600 is directed for stimulating control of stutter and is designed as a modification of device 100. Device 600 includes a moving sensation part 602, a portion 604 of which is arranged to contact a user of device 600. Moving sensation part 602 is configured to exert a tactile sensation of a move along a path 606 over the user body in a continuous pattern for helping the user control their flow of speech. Path 602 is a closed curve disposed at the bottom 608 of device 600, such that when portion 604 protrudes therefrom (from path 606) to contact the user body and is activated to move there along (along path 606), the user feels a tactile sensation of movement. Device 600 may be designed with different paths and patterns as exemplified in FIG. 8. FIG. 8 illustrates examples of paths that can be used in conjunction with a stutter control device, constructed and operative in accordance with further embodiments of the present invention. The examples in FIG. 8 include reciprocating (side-to-side, back-and-forth, linear) pattern 801, closed curved paths such as figure-eight path 803, ellipsoidal path 805, undefined curved path 807, Helical paths 809 and 811, and spiraling path all of which can be an open curved path if cut anywhere (with back and forth motions there along), such as wave path 815. The paths may also be closed polygonal path such as path 817, open polygonal path such as path 819 and changing path such as path 821. The paths may be defined by a simple mechanism that is designed to advance portion 604 along a permanent rail disposed at bottom 608, or determined by a mechanism that is enabled to move portion 604 anywhere in an open bottom 608. In the latter case, a different path may be selected by the user or chosen by a control element of device 600, and even a random path may be determined randomly. Variation of the path can contribute to elimination of user habituation to the routine stimulation and thereby contributing to maintaining effective stimulating sensation.

The speed by which part 604 moves at a continuous pattern, may be a steady speed pattern or a varying speed pattern, and may be varied according to a predetermined program, or at a randomly varying speed pattern. Variation of the speed can contribute to elimination of user habituation to the routine stimulation and thereby help maintain effective stimulating sensation.

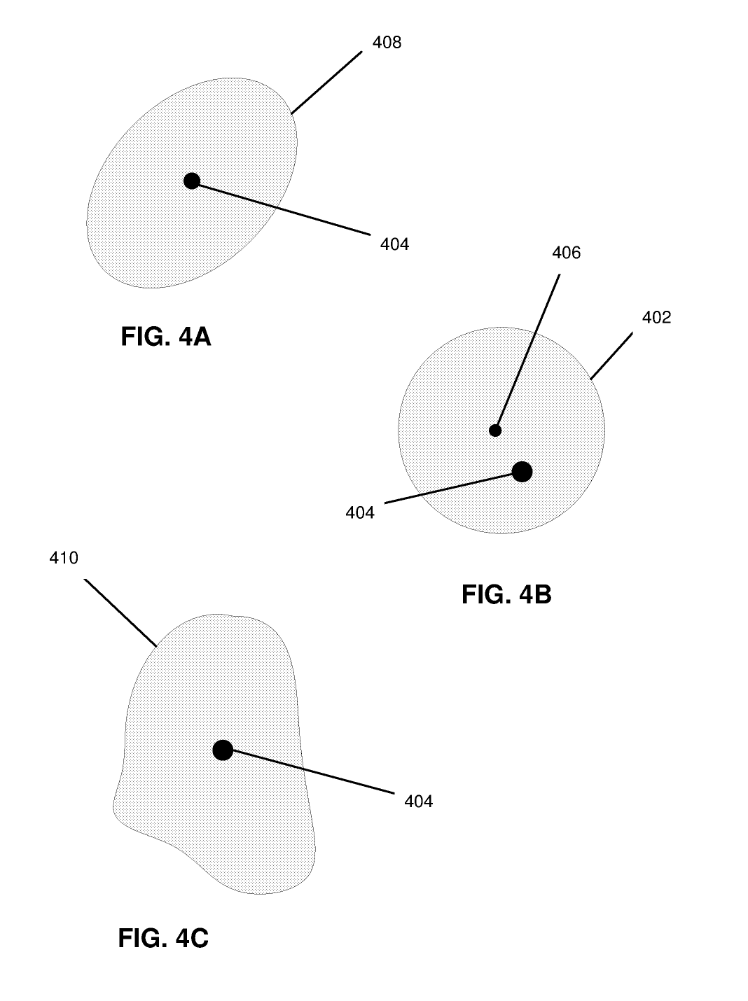

In addition, the pressure exerted by part 604 on the user body when moved at continuous pattern, may be a steady pressure pattern or a varying pressure pattern, and may be varied according to a predetermined program, or at a randomly varying pressure pattern. Variation of the pressure can contribute to elimination of user habituation to the routine stimulation and thereby help maintain effective stimulating sensation. Referring back to the embodiment of FIG. 3, the stutter control device was described with moving sensation part 312 moving along an elongated path as in FIG. 1, or in a “path” which is in a fixed location. Part 312 is a moving sensation part such as a rolling element which is placed in rolling contact with the user, and is moved along a path (or in a fixed location). The moving sensation may be created by the movement of part 312 (such as a simple wheel) along a path, by the frequent or repeated change in direction of movement, or by the mere gradual pressure change, due to an optional asymmetry of rotating part 312. Such moving sensation part can rotate about an asymmetrical axis to create a dynamically changing pressure over a portion of the user body. Further asymmetric cross-sectional profiles of such a rotating part, are shown in FIGS. 4a to 4c. FIGS. 4a to 4c illustrate examples of alternative tactile interfaces of the embodiment of FIG. 3. FIG. 4b illustrates a round profile 402, whose axis of rotation 404 is displaced with regard to its center 406. FIG. 4a illustrates an aspheric profile 408, which is a modified (imperfect) ellipsoidal profile, in comparison to the ellipsoidal profile of part 312. FIG. 4c is a unevenly curved profile 410. It is preferable to use a smooth closed curve profile to avoid excess rubbing of the skin or clothes by the drag caused by sharp edges. However, it may be preferable to use a closed curve profile whose curvature has no cusps, valleys or depressions that may interrupt contact with the skin and thereby derogate the continuous stimulation, such as would be provided by a closed convex curve profile. In addition, any such rolling element may be lubricated over its tactile interfacing surface for reducing skin drag and irritation.

Device 600 may be operated with a continuous pattern which is repeated at a frequency ranging from 0.25 seconds per cycle to 5 seconds per cycle (240-12 CPM), wherein each cycle could mean the completing a closed path (or one back and forth movement along a section of a open path, or the completing of a speed change cycle, or the completing of a pressure change cycle. Preferably, the frequency can be in the range of 0.5 seconds per cycle to 3 seconds per cycle (120-20 CPM), and further preferably ranging from 1 seconds per cycle to 2 seconds per cycle (60-30 CPM).

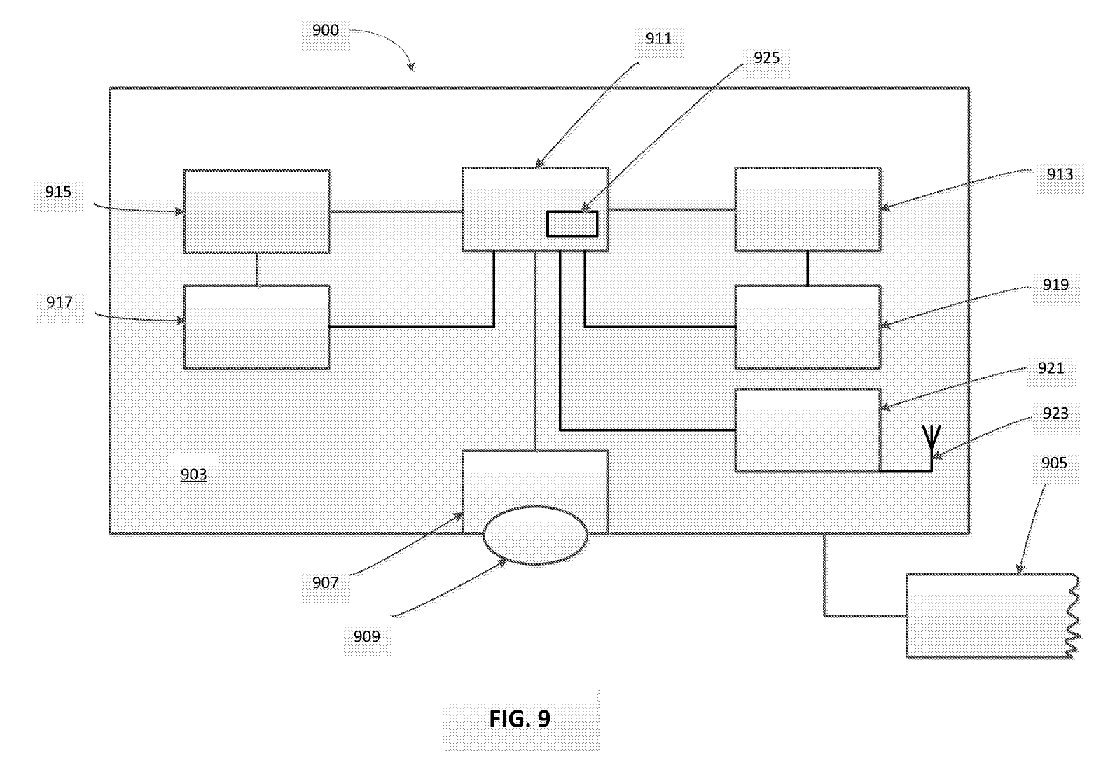

The manipulation and control of the speed and pressure of the moving sensation part, as well as performance recording, analysis, or feedback and communication to external devices, may be conducted with an adequate user control interface and complementary means. Reference is now made to FIG. 9, which is a block diagram of a stutter control device 900, constructed and operative in accordance with a further embodiment of the present invention. Device 900 includes housing or frame 903, which is fastened to the user body by fastener 905. Device 900 further includes moving sensation part 907, which incorporates tactile element 909, controller 911, user control interface 913, optional voice recognition module 915, optional speech sensor 917, optional display interface 919, and optional communication module 921. Controller 911 includes a processor for controlling and performing all the necessary computing and managing operations of device 900. Controller 911 is connected to moving sensation part 907, user control interface 913, optional voice recognition module 915, optional speech sensor 917, optional display interface 919, and optional communication module 921. It is noted that device 900 includes features that are common to communication or interface devices, such as handheld (or wrist or otherwise worn) tablets and cell phones (such as features 911, 913, 917, 919, 921, 923, and 925). Accordingly, device 900 may be incorporated into or integral with such communication or interface devices, or provided as an add-on device in (wired or wireless) communication with such communication or interface devices and typically managed or controlled through such a communication or interface device.

Optional display interface 919 may be integrated with user control interface 913 or connect therewith. Optional speech sensor 917 may be integrated with optional voice recognition module 915 or connect therewith, Controller 911 is connected to moving sensation part 907 and controls its activation and deactivation, and optionally the speed, pressure and/or path of moving sensation part 907, if any or all of these allow variations. User control interface 913 may be used to allow the user or the therapist to control at least one of: the speed, the pressure, and the path of moving sensation part 907, as well as all optional operations of the elements of device 900. In case controller 911 incorporates preprogrammed speed, pressure and/or path plans, user control interface 913 may be used to allow the user or the therapist to control at least one of: a predetermined speed program selection, a predetermined pressure program selection, and/or a predetermined path program selection of moving sensation part 907, in addition to further optional operations.

User control interface 913 can be managed by a speech therapist as part of a therapy session or to let the therapist program the predetermined parameters for further application by the user as part of the therapeutic scheme suiting the user speech control progress. The actual performance data of device 900 (e.g.: time of use, speed, paths pressures and cycle frequencies used, etc.) can be recorded and stored, such as in a memory storage module 925 of controller 911, and displayed—such as by optional display interface 919, or relayed or communicated by optional communication module 921 to some external storage or display for further analysis by therapists and for monitoring of the user progress. Optional communication module 921 can communicate with external devices by wired means or wirelessly, as represented by antenna 923.

Optional voice recognition module 915 is connected to controller 911 and is installed for identifying when the user talks, requiring a speech sensor such as a microphone, which can be integral with voice recognition module 915 or a separate unit such as optional speech sensor 917. Voice recognition module 915 includes speech analysis means and provides this information to controller 911, which activates moving sensation part 907 (or device 900 altogether) only when the user talks. Recording of the user speech in conjunction with the use of device 900 can be done by the use of speech sensor 917, and stored in memory storage module 925 for further play by display interface 919 which includes adequate sound playing means or forwarded by communication module 921 to external storage and sound playing means for further monitoring and analysis, as a particular form of the data stored or communicated as mentioned above. The voice analysis in real time or thereafter may be performed by controller 911 and displayed (or indicated by some sound or other tactile interface) by user display interface 919 to the user or the therapist. The indication of stuttering or its degree, or the measure by which it is controlled, and its dynamic change, especially if presented to the user in real time, can provide a feedback of the user progress in response to use or mode of use of device 900.

Fastener 905 includes at least one fastening element for attaching device 900 to the user. Preferably, the at least one fastening element is operative for attaching device 900 to the user in different body regions, such as to the arm, the chin, the waist, and so forth as mentioned above with respect to device 100. Accordingly, the at least one fastening element of fastener 905 may be a clip, a belt, a removable skin-adhesive, a removable cloth-adhesive, and a band.

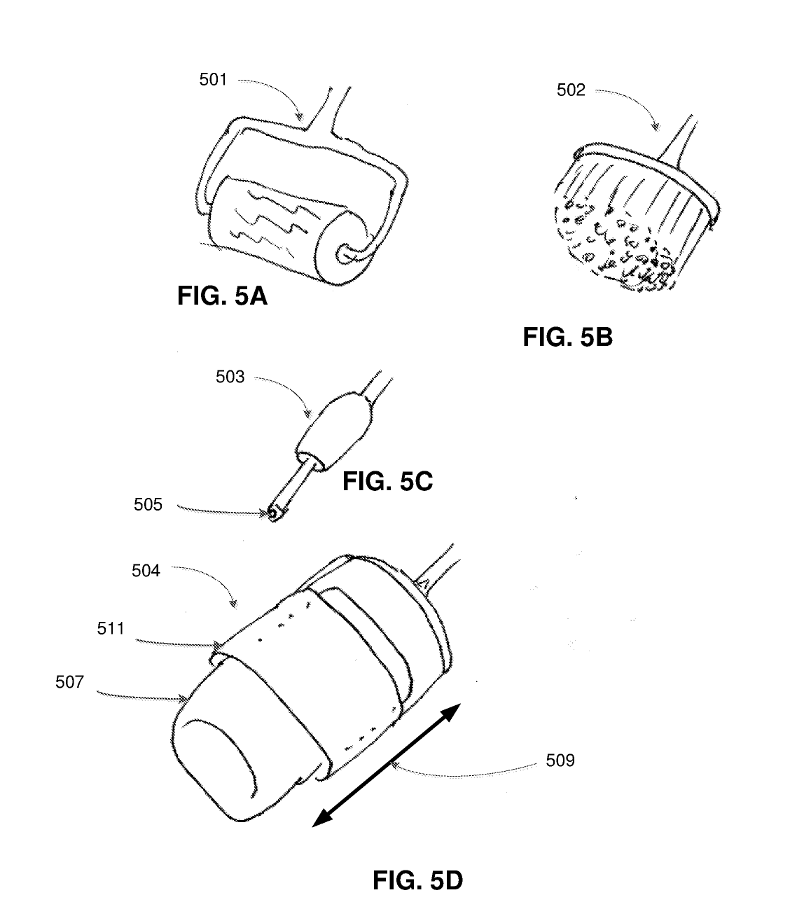

The moving sensation part is not limited to rolling, inflated or bulging elements, such as those described above and other examples that are effective to create the moving sensation, may be implemented. Reference is now made to FIGS. 5a to 5d, which illustrate further examples of tactile interfaces for inspiring touch or movement sensation, constructed and operative in accordance with further embodiments of the present invention. FIG. 5a illustrates a rolling element 501 similar to the one described with reference to FIGS. 1-3. An alternative embodiment is a gentle-stroke element, which is in caressing contact with the user skin, when moved along a path over the user body. FIG. 5b illustrates an example of such a gentle-stroke element, in the form of brush 502. FIG. 5c illustrates an air jet element 503 operative for blowing continuous air jet through nostril or nozzle 505 over the user skin, when air jet element 503 is moved along a path over the user body. FIG. 5d illustrates a pressure exerting element 504 which incorporates a rounded protuberant projection which is selectively and retractably extendable along arrow 509 from frame 511.

The creation of a moving sensation along a path over the body can be created without requiring the actual movement of a moving part along the path. In reference to FIG. 7, there is shown a bottom view of stutter control device 700, constructed and operative in accordance with another embodiment of the present invention. Device 700 includes moving sensation part 701 extending over the entire bottom part of device 700, which is in contact with the user body (the skin or over a cloth). Moving sensation part 701 includes an array in the form of a matrix of stationary touch sensation elements 703, appearing as rounded objects arranged in a grid (over the entirety of part 701—only some of elements 703 are illustrated, any other suitable arrangement can be chosen). Each stationary touch sensation element 703 is disposed in a fixed location. Touch sensation elements 703 are selectively activated along a path 704, denoted by blackened rounded objects 705, to create a moving sensation. The activation of touch sensation elements 703 is performed one at the time, only along path 704 and in succession along path 704, thereby creating a moving sensation. Touch sensation elements can be of any form described above with respect to a moving part, i.e.: a rolling, bulging, retractably projecting, air jet generating, or gently stroking element.

While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. A device for stimulating a user in order to control speech stutter, comprising:
a moving sensation part, a portion of which is arranged to contact a user of the device, configured to exert a tactile sensation of moving over the user's body in a continuous pattern for helping users to control their flow of speech.

2. The device according to claim 1, wherein said continuous pattern comprises moving along at least one path selected from the group consisting of:
a reciprocating (side-to-side, back-and-forth, linear) pattern;
a closed curved path;
an open curved path;
a wave path;
a figure-eight path;
an ellipsoidal path;
a helical path;
a spiraling path;
a closed polygonal path;
an open polygonal path;
a changing path; and
a random path,

3. The device according to claim 1, wherein speed of said continuous pattern comprises at least one selected from the group consisting of:
a steady speed pattern;
a varying speed pattern, varying according to a predetermined program; and
a randomly varying speed pattern.

4. The device according to claim 1, wherein pressure of said continuous pattern comprises at least one selected from the group consisting of
a steady pressure pattern;
a varying pressure pattern, varying according to a predetermined program; and
a randomly varying pressure pattern.

5. The device according to claim 4, wherein path of said moving sensation part is in a fixed location, and wherein the moving sensation is created by at least one of gradual pressure change; gradual speed change; and an alternating rolling direction of a rolling element of said moving sensation part.

6. The device according to claim 1, wherein said moving sensation part comprises rolling element comprising at least one selected from the list consisting of:
a rolling element in rolling contact with the user which is moved along said path; and
a rolling element which rotates about an asymmetrical axis to create a dynamically changing pressure over a portion of the user body.

7. (canceled)

8. (canceled)

9. The device according to claim 6, wherein said rolling element comprises a cross sectional profile selected from the group consisting of:
a round profile;
an ellipsoidal profile;
an aspheric profile;
a smooth closed curve profile;
a closed convex curve profile;
a closed curve profile whose curvature has no cusps, valleys or depressions that interrupt contact with the skin; and
an unevenly curved profile.

10. The device according to claim 1, wherein a controller is configured to control at least one operation of:
speed of said moving sensation part;
pressure of said moving sensation part;
path of said moving sensation part;
a predetermined speed program selection of said moving sensation part;
a predetermined pressure program selection of said moving sensation part; and
a predetermined path program selection of said moving sensation part;
storing usage data;
recording user speech;
analyzing stored or recorded data;
recording user performance in conjunction with usage data;
displaying stored, recorded or analyzed data; and
communicating with external devices.

11. The device according to claim 10, further comprising user control interface for managing at least one of said operations.

12. The device according to claim 1, wherein said moving sensation part comprises a gentle-stroke element in caressing contact with the user skin which is moved along said path.

13. The device according to claim 1, wherein said gentle stroke element comprises a brush.

14. The device according to claim 1, wherein said moving sensation part comprises an air jet element operative for blowing continuous air jet over the user skin, wherein said air jet element is moved along, said path.

15. The device according to claim 1, wherein said moving sensation part comprises a matrix of stationary touch sensation elements in fixed locations and wherein said touch sensation elements are selectively activated along said path to create a moving sensation.

16. The device according to claim 15, wherein said stationary touch sensation elements comprise at least one of:
gentle stroke elements;
air jet elements;
rolling elements;
bulging elements;
inflating elements; and
pressure exerting elements.

17. The device according to claim 1, wherein said continuous pattern is repeated at a frequency ranging from 0.25 seconds per cycle to 5 seconds per cycle (240-12 CPM), wherein each cycle includes at least one of: completing a dosed path or back and forth movement along a section of an open path; completing a speed change cycle; and completing a pressure change cycle.

18. The device according to claim 17, wherein said frequency ranges from 0.5 seconds per cycle to 3 seconds per cycle (120-20 CPM).

19. The device according to claim 17, wherein said frequency ranges from 1 second per cycle to 2 seconds per cycle (60-30 CPM).

20. The device according to claim 1, further comprising voice recognition for identifying when said user talks and wherein said device is controlled to be activated only when said user talks.

21. The device according to claim 1, further comprising at least one fastening element for attaching said device to the user.

22. (canceled)

23. (canceled)

24. The device according to claim 1, wherein said moving part is incorporated into a mobile object selected from the group consisting of:
a watch;
a handheld interface device; and
a handheld communication device.
